(19) United States
(12) Patent Application Publication
(10) Pub. No.: US 2018/0256606 A1
(43) Pub. Date: Sep. 13, 2018

(54) MULTILAYER DOSAGE FORMS, WHICH CONTAIN ACTIVE SUBSTANCES AND WHICH COMPRISE A NEUTRAL CORE, AND AN INNER AND OUTER COATING CONSISTING OF METHACRYLATE COPOLYMERS AND METHACRYLATE MONOMERS

(71) Applicant: Evonik Roehm GmbH, Darmstadt (DE)

(72) Inventors: Hans-Ulrich PETEREIT, Darmstadt (DE); Markus RUDOLPH, Neu-lsenburg (DE); Jennifer DRESSMAN, Frankfurt (DE); Thomas BECKERT, Warthausen (DE)

(73) Assignee: Evonik Roehm GmbH, Darmstadt (DE)

(21) Appl. No.: 15/975,090

(22) Filed: May 9, 2018

Related U.S. Application Data
(63) Continuation of application No. 10/532,831, filed on Mar. 9, 2006, now abandoned.

(30) Foreign Application Priority Data
Oct. 29, 2002 (DE) 102 50 543.8

(51) Int. Cl. A61K 31/58; A61K 9/50; A61K 9/16

(52) U.S. Cl. CPC A61K 31/58; A61K 9/2846; A61K 9/1635; A61K 9/5078

(57) ABSTRACT

The invention relates to a multilayer dosage form comprised of: a) a neutral core; b) an inner coating consisting of a methacrylate copolymer; c) an outer coating consisting of a copolymer of which 40 to 95% by weight is composed of radically polymerized C₁ to C₄ alkyl esters of acrylic acid or of methacrylic acid and of which 5 to 60% by weight is composed of (meth)acrylate monomers having an anionic group in the alkyl radical. The invention is characterized in that the inner coating is essentially comprised of a methacrylate copolymer, of which at least up to 90% by weight consists of (meth)acrylate monomers with neutral radicals, which, in accordance with DIN 53 787, has a minimum film formation temperature of no higher than 30° C., and which contains the pharmaceutical active substance in bound form.

This application is a continuation of U.S. application Ser. No. 10/532,831, filed on Apr. 26, 2005, with a § 371(c) date of Mar. 9, 2006; which is a national stage application of International Patent Application No. PCT/EP03/09800, filed on Sep. 4, 2003, designating the United States and published in German on May 13, 2004 as publication WO 2004/039357 A1; which claims priority to German Patent Application No. 102 50 543.8, filed on Oct. 29, 2002. The contents of the afore-mentioned patent applications are incorporated herein by their entirety.

The invention relates to a multilayer dosage form with neutral methacrylate copolymer as binder for the active substance.

### PRIOR ART

The use of so-called neutral methacrylate copolymers, which are methacrylate copolymers which consist predominantly of (at least 95%) (meth)acrylate monomers with neutral radicals, such as methyl methacrylate or ethyl acrylate, as coating agents and binders for dosage forms with delayed release of active substances has been known for a long time.

Uses in mixtures with anionic dispersions are described for example in EP-A 152 038, EP-A 208 213 or EP-A 617 972.

WO 01/68767 describes a dispersion suitable for the use as coating agent and binder for dosage forms, having a solids content of 10-70% by weight consisting of
a) 90 to 99% by weight of a methacrylate copolymer which consists of at least 90% by weight of (meth)acrylate monomers with neutral radicals, and a glass transition temperature Tg of from −20° C. to +20° C. determined by the DSC method, and b) 1-10% by weight of a nonionic emulsifier with an HLB of from 15.2 to 17.3.

The use of the specific emulsifiers disclosed in WO 01/68767 allows, while retaining the stability of the dispersion and of its particle size distribution, pharmaceutical formulations to be produced therefrom, with which phase separation with the formation of crystal structures owing to the emulsifier does not occur.

WO 01/68767 further mentions that multilayer coating system layers can be produced. For example, a core which comprises for example basic or water-sensitive active substances can be provided with a sealing layer of another coating material such as cellulose ether, cellulose ester, cationic polymethacrylates (such as Eudragit® E 100, -RL 100, -RS 100, Röhm GmbH), before the coating agent of the invention is applied. Likewise, further coatings, for example having an odor- or taste-masking effect or having a pleasing coloring or gloss effect, can be applied subsequently.

A typical methacrylate copolymer according to WO 01/68767 may be composed for example of 25-35% by weight methyl methacrylate and 75 to 65% by weight ethyl acrylate. It is also possible where appropriate for small comonomer contents of other vinyl monomers to be present.

Multilayer dosage forms have been known for some time. WO 01/68058 describes for example the use of a multilayer dosage form which is essentially composed of
a) a core with a pharmaceutical active substance
b) an inner coating of a copolymer or of a mixture of copolymers composed of 85 to 98% by weight free-radical polymerized C1- to C4-alkyl esters of acrylic or of methacrylic acid and 15 to 2% by weight (meth)acrylate monomers having a quaternary ammonium group in the alkyl radical, and
c) an outer coating of a copolymer which is composed of 75 to 95% by weight free-radical polymerized C₁- to C₄-alkyl esters of acrylic or of methacrylic acid and 5 to 25% by weight (meth)acrylate monomers having an anionic group in the alkyl radical
   for producing a dosage form for which, in the USP release test two hours at pH 1.2 and a subsequent change in the buffer to pH 7.0, the release of the active substance present is less than 5% in the period up to 2.0 hours after the start of the test and 30 to 80% at the time eight hours after the start of the test.

### Problem and Solution

The present invention starts from WO 01/68767. The multilayer dosage form described therein permits the adjustment of variable release profiles and a delivery of active substance which is precise and reproducible under defined conditions.

Production thereof is comparatively complicated due to the multilayer structure which is to be produced in a plurality of operations.

In addition, after the outer coating layer has dissolved off, it is not completely precluded that, depending on the layer thicknesses adjusted, the compositions, the particular active substance and its concentration, there may be interactions between the inner coating layer and the active substance molecules released slowly from the core. This appears to be particularly the case with active substances having polar or ionic groups, which may enter into interactions with the positively charged quaternary ammonium groups of the (meth)acrylate copolymers or the chloride counter-ions thereof in the molecule.

A further problem is that the active substance release characteristics are evidently influenced by the ionic strength of the surrounding medium. Since in particular oral dosage forms are frequently taken with water, and the ionic strength in the stomach and intestine are also always subject for example through food intake to certain variations, the dosage forms are exposed to varying ionic strengths in vivo. This may, in vivo, lead to active substance release characteristics which are not always reproducible. Desirable dosage forms therefore have active substance release characteristics which are very substantially uninfluenced by the ionic strength of the surrounding medium.

An additional factor is that the inner coating layer must usually be formulated with the aid of plasticizers in order to ensure adequate flexibility of the films. The use of release agents such as, for example, talc or glycerol monostearate is also usually unavoidable in order to prevent adhesion of the coated units during or after application of the inner coating layer.

The problem was therefore regarded as being the development of a multilayer dosage form which, similar to that of WO 01/68767, allows the adjustment of variable release profiles and a delivery of active substance which is precise and reproducible even with different ionic strengths of the surrounding medium. It should, however, be possible to produce the multilayer dosage form comparatively more simply. In addition, it should be possible for possible interactions between the active substance present and polymeric coating agents or plasticizers coming into contact with the active substance to be kept small or avoided.

The problem is solved by a

multilayer dosage form composed of
a) a neutral core,
b) an inner coating of a methacrylate copolymer
c) an outer coating of a copolymer which is composed of 40 to 95% by weight free-radical polymerized C₁- to C₄-alkyl esters of acrylic or of methacrylic acid and 5 to 60% by weight (meth)acrylate monomers having an anionic group in the alkyl radical,

characterized in that

the inner coating consists substantially of a methacrylate copolymer which is composed of at least 90% by weight of (meth)acrylate monomers having neutral radicals, has a minimum film-forming temperature as specified in DIN 53 787 not exceeding 30° C., and comprises the pharmaceutical active substance in bound form.

Compared with the multilayer dosage form disclosed in WO 01/68767, the dosage form of the invention can be produced more easily because the active substance can be applied in one working step with the inner polymer coating. The use of a methacrylate copolymer which is composed of at least 90% by weight of (meth)acrylate monomers having neutral radicals, has a minimum film-forming temperature as specified in DIN 53 787 not exceeding 30° C., makes it possible to dispense substantially or even completely with excipients such as plasticizers or release agents. In an advantageous manner and non-predictably, dissolution of the outer coating in the colon is followed by a similarly slow release of the active substance bound in the polymer as is possible in WO 01/68767 with an active substance bound in the core and with a coating of (meth)acrylate copolymers with quaternary amino groups. An important advantage of the dosage form of the invention is that the release of active substance is virtually uninfluenced by the ionic strength at constant pH in a hypotonic and an isotonic medium.

### MODE OF OPERATION OF THE INVENTION

The invention relates to a

multilayer dosage form which, in the USP release test two hours at pH 1.2 and a subsequent change in the buffer to a pH of at least 6.8, releases a pharmaceutical active substance present the active substance present to the extent of less than 5% in the period up to 2.0 hours after the start of the test and 30 to at least 80% at the time eight hours after the start of the test

and is composed of
a) a neutral core,
b) an inner coating of a methacrylate copolymer
c) an outer coating of a copolymer which is composed of 75 to 95% by weight free-radical polymerized C₁- to C₄-alkyl esters of acrylic or of methacrylic acid and 5 to 60% by weight (meth)acrylate monomers having an anionic group in the alkyl radical,

characterized in that

the inner coating consists substantially of a methacrylate copolymer which is composed of at least 90% by weight of (meth)acrylate monomers having neutral radicals, has a minimum film-forming temperature as specified in DIN 53 787 not exceeding 30° C., and comprises the pharmaceutical active substance in bound form.

Cores a)

Carriers or neutral cores for the coatings are tablets, granules, pellets, crystals of regular or irregular shape. The size of granules, pellets or crystals is usually between 0.01 and 2.5 mm, and that of tablets between 2.5 and 30.0 mm.

The cores may comprise further pharmaceutical excipients: binders, such as lactose, cellulose and derivatives thereof, polyvinylpyrrolidone (PVP), humectants, disintegration promoters, lubricants, disintegrants, starch and derivatives thereof, sugar solubilizers or others.

Inner Coating b)

The inner coating b) consists substantially of a methacrylate copolymer which consists of at least 90% by weight of (meth)acrylate monomers with neutral radicals and has a minimum film-forming temperature as specified in DIN 53 787 not exceeding 30° C., particularly preferably not exceeding 25° C., with a pharmaceutical active substance bound therein.

The layer thickness of the inner coating can preferably be between 10 and 300 μm.

Methacrylate Copolymer for the Inner Coating b)

The methacrylate copolymer for the inner coating b) consists of at least 90, in particular 95, preferably 97, in particular 99, particularly preferably 100% by weight of (meth)acrylate monomers with neutral radicals, in particular C₁- to C₄-alkyl radicals.

Examples of suitable monomers are methyl methacrylate, ethyl methacrylate, butyl methacrylate, methyl acrylate, ethyl acrylate, butyl acrylate. Methyl methacrylate, ethyl acrylate and methyl acrylate are preferred.

The polymers which are neutral per se may comprise small amounts of methacrylic acid or acrylic acid which, although they make virtually no change in the insolubility of the polymer in water, may influence the swelling and permit pH-dependent control of permeability.

Other vinylically polymerizable monomers, especially (meth)acrylate monomers with polar or ionic radicals, e.g. methacrylic acid or acrylic acid, may be present in small amounts, not exceeding 10, preferably not exceeding 5, particularly preferably not exceeding 3 or not exceeding 1% by weight.

The (meth)acrylate copolymer has a minimum film-forming temperature as specified in DIN 53 787 not exceeding 30° C.

The methacrylate copolymer may preferably have a glass transition temperature Tg of from −25° C. to +20° C., preferably −10° C. to 0° C., determined by the DSC method (ISO 11357).

The methacrylate copolymer of the inner coating may for example be polymerized from 25-35% by weight methyl methacrylate, 75 to 65% by weight ethyl acrylate and not more than 1% by weight methacrylic acid, where the proportionate amounts add up to 100% by weight.

The (meth)acrylate copolymer for the inner coating b) may be in the form of or processed as organic solution or as dispersion.

The (meth)acrylate copolymer for the inner coating b) is preferably employed in the form of a dispersion with a solids content of 10-70% by weight.

The corresponding dispersion particularly preferably comprises from 1 to 10, preferably 2 to 8, particularly preferably 4 to 6, % by weight, based on the solids content, of a nonionic emulsifier with an HLB of from 15.7 to 19.5. A suitable example is polyoxyethylene 100 isononylphenol (HLB about 19.1).

Emulsifiers

Emulsifiers control the progress of the emulsion polymerization process by making the chain-building reaction of the emulsified monomers possible in the aqueous phase. They are therefore an auxiliary which is necessary for production and determine the properties of the dispersion. They cannot normally be exchanged without fundamentally changing relevant properties of the dispersion.

The HLB is a measure which was introduced by Griffin in 1950 of the hydrophilicity or lipophilicity of nonionic surfactants. It can be determined experimentally by the phenol titration method of Marszall, cf. “Parümerie, Kosmetik”, Volume 60, 1979, pp. 444-448; further references in Römpp, Chemie-Lexikon, 8th edition, 1983, page 1750. See also, for example, U.S. Pat. No. 4,795,643 (Seth)).

An HLB (hydrophilic/lipophilic balance) can be determined accurately only for nonionic emulsifiers. With anionic emulsifiers it is possible to determine this value by calculation, but it is virtually always above or far above 20.

The HLB values of the emulsifiers have a distinct effect on the crystallization of the emulsifier. In the ideal case, these values are between 15.7 and 16.2. Above the claimed range, the emulsifiers crystallize out after drying. Emulsifiers with an HLB below the claimed range are unable to stabilize the dispersion sufficiently, as is evident from pronounced coagulation. The HLB values were either taken from the literature (Fiedler: Lexikon der Hilfsstoffe) or calculated as described by W. C. Griffin (offprint from Parfümerie and Kosmetik 64, 311-314, 316 (1983); Hüthig Verlag, Heidelberg/Pharmind Ind. 60 No. 1 (1998); dielectricity thermal analysis).

The emulsifier should be toxicologically acceptable and therefore preferably nonionic emulsifiers.

Suitable classes of emulsifiers are ethoxylated fatty acid esters or ethers, ethoxylated sorbitan ethers, ethoxylated alkylphenols, glycerol esters or sugar esters or wax derivatives.

Examples of suitable emulsifiers are polyoxyethylene glycerol monolaurate, polyoxyethylene glycerol monostearate, polyoxyethylene 20 cetylstearate, polyoxyethylene 25 cetylstearate, polyoxyethylene 25 oxypropylene monostearate, polyoxyethylene 20 sorbitan monopalmitate, polyoxyethylene 16 tert-octylphenol, polyoxyethylene 20 cetyl ether, polyethylene glycol 1000 monocetyl ether, ethoxylated castor oil, polyoxyethylene sorbital-wool wax derivatives, polyoxyethylene 25 propylene glycol stearate and polyoxyethylene sorbitol ester.

Preference is given to polyoxyethylene 25 cetylstearate, polyoxyethylene 20 sorbitan monopalmitate, polyoxyethylene 16 tert-octylphenol and polyoxyethylene 20 cetyl ether.

Production of a Dispersion

A dispersion is obtained in a manner known per se by aqueous emulsion polymerization in a batch or feed process, semicontinuously or else continuously (concerning this, see, for example, DE 195 03 099 A1).

Free-radical polymerization of the monomers in the presence of the emulsifier takes place using free radical-forming water-soluble polymerization initiators, with the radical formation possibly taking place thermally or by redox processes. Molecular weight regulators are added where appropriate to adjust the molecular masses. Emulsions polymers are normally produced in concentrations between 10 and 70% by weight. A favorable solids content is 30-50% by weight. Batchwise production normally takes place in stirred tank reactors.

For production by simple batch production, all the monomers according to the desired copolymer composition are introduced together with the emulsifier, initiators, regulators and other aids together with water into a reaction tank and dissolved or dispersed therein. The polymer chain reaction is initiated and carried out by activating the initiator (raising the temperature, adding the redox agent). During this there is formation of the known latex particles consisting of polymer chains.

It is possible to add antifoam emulsion and stabilizers to the dispersion.

Production of the Dosage Form of the Invention

Spray Application

The application processes necessary for implementing the invention correspond to the prior art and are described for example in the following textbooks:
Bauer, Lehmann, Osterwald, Rothgang, “Uberzogene Arzneiformen” Wissenschaftliche Verlagsgesellschaft mbH Stuttgart, Chapter 7, pages 165-196
Bauer, Lehmann, Osterwald, Rothgang, “Coated Pharmaceutical Dosage Forms” CRS Press 1988, Chapter 7
J. W. McGinity (Ed.), Aqueous Coatings for Pharmaceutical Dosage Forms, Marcel Dekker Inc., 1997
K. Lehmann et al., “Practical Course in Film Coating of Pharmaceutical Dosage Forms with Eudragit®”, Röhm GmbH & Co. KG., 2001
M. Dombrow (Ed.) Microcapsules and Nanoparticles in Medicine and Pharmacy, CRS Press, 1992

Further processing to oral dosage forms

Usual prior art processes are used. Details are to be found in the relevant textbooks, e.g.:
Voigt, R. (1984): Lehrbuch der pharmazeutischen Technologie; Verlag Chemie Weinheim-Beerfield Beach/Florida-Basle.
Sucker, H., Fuchs, P., Speiser, P.: Pharmazeutische Technologie, Georg Thieme Verlag Stuttgart (1991), especially Chapters 15 and 16, pages 626-642.
Gennaro, A. R. (Editor), Remington's Pharmaceutical Sciences, Mack Publishing Co., Easton Pa. (1985), Chapter 88, pages 1567-1573.
List, P. H. (1982): Arzneiformenlehre, Wissenschaftliche Verlagsgesellschaft mbH, Stuttgart.

Particularly important in this connection are compression to tablets and packing in capsules. Properties relevant for administration, required tests and specifications are listed in pharmacopeias.

Binding of the Active Substance

The binding of the active substance preferably takes place by aqueous spraying of an active substance-containing (meth)acrylate copolymer dispersion onto the cores a), e.g. sucrose pellets, with binding of the active substance after evaporation or sublimation of the water. The product temperature during the spray application can in this case be for example 20 to 40, preferably 25 to 35° C.

One variant of the process is the so-called powder layering process in which the (meth)acrylate copolymer dispersion is sprayed and, during this, the active substance is added in powder form.

It is usually possible to dispense with release agents such as, for example, talc or with addition of plasticizer during the processing of the active substance-containing (meth)acrylate copolymer dispersion.

The processing of the active substance can preferably take place by stirring into water with initially vigorous mixing, e.g. by mixing for 5 to 15 minutes for example with a high-speed mixer (homogenizer). The suspension or solution obtained in this way can then be added to the (meth)acrylate copolymer dispersion. The mixture should expediently and preferably also be agitated continuously during the spraying process. It is additionally possible for a water-soluble active substance to be put in dissolved form into the polymer dispersion and subsequently sprayed on.

The active substance is present in the copolymer of the inner coating b) either in crystalline form (solid dispersion) or in dissolved form (solid solution).

The active substance/polymer ratio in the inner layer can be from 20:1 to 1:20, preferably 1:1 to 1:3.

Pharmaceutical Active Substances

The dosage form of the invention is suitable for administering in principle any pharmaceutical active substances which are to be released preferably in the small intestine and/or colon and, in particular, those which can advantageously be administered in delayed-release form, such as antidiabetics, analgesics, antiinflammatory agents, antirheumatic agents, anti-hypotensives, antihypertensives, psycho-pharma-ceuticals, tranquilizers, antiemetics, muscle relaxants, glucocorticoids, agents for treating ulcerative colitis or Crohn's disease, antiallergics, antibiotics, antiepileptics, anticoagulants, anti-mycotics, antitussives, arteriosclerosis remedies, diuretics, enzymes, enzyme inhibitors, gout remedies, hormones and their inhibitors, cardiac glycosides, immunotherapeutics and cytokines, laxatives, lipid-lowering agents, migraine remedies, mineral preparations, otologicals, antiparkinson agents, thyroid therapeutics, spasmolytics, platelet aggregation inhibitors, vitamins, cytostatics and metastasis inhibitors, phytopharmaceuticals, chemo-therapeutics and amino acids.

Examples of suitable active substances are acarbose, beta-receptor blockers, non-steroidal antiinflammatory drugs, cardiac glycosides, acetylsalicylic acid, virustatics, aclarubicin, acyclovir, cisplatin, actinomycin, alpha- and beta-sympathomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclo-phosphamide, ciclosporin, cyproterone, cytarabine, dapiprazole, desogestrel, desonide, dihydralazine, diltiazem, ergot alkaloids, dimenhydrinate, dimethyl sulfoxide, dimethicone, dipyridarnoi, domperidone and domperidane derivatives, dopamine, doxazosine, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium channel blockers, irinotecan, modafinil, orlistat, peptide antibiotics, phenytoin, riluzoles, risedronate, sildenafil, topiramate, macrolide antibiotics, estrogen and estrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, flunarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, St John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramin, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixen, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novaminsulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocin, pentifylline, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexol, pravastatin, prazosin, procaine, promazine, propiverine, propranolol, propyphenazone, prostaglandins, protionamide, proxyphylline, quetiapine, quinapril, quinaprilate, ramipril, ranitidine, reproterol, reserpine, ribavirin, rifampicin, risperidone, ritonavir, ropinirol, roxatidine, roxithromycin, ruscogenin, rutoside and rutoside derivatives, sabadilla, salbutamol, salmeterol, scopolamine, selegiline, sertaconazole, sertindol, sertralion, silicates, simvastatin, sitosterol, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiam, sumatriptan, suxamethonium chloride, tacrine, tacrolimus, taliolol, tamoxifen, taurolidine, tazaroten, temazepam, teniposide, tenoxicam, terazosin, terbinafine, terbutaline, terfenadine, terlipressin, tertatolol, tetracyclines, tetryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, anti6strogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutin, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloxazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, zotepine and the like.

Examples of particularly preferred active substances are agents for treating ulcerative colitis or Crohn's disease such as salicylates, e.g. 5-aminosalicylic acid, 4-aminosalicylic acid, olsalazine, balsalazine, sulfasalazine, corticosteroids such as budesonide, prednisolone, methylprednisolone, prednisone, dexamethasone, hydrocortisone, triamcinolone, antiasthmatics such as theophylline and salbutamol, analgesics such as tramadol, morphine, codeine, proton pump inhibitors such as omeprazole, virustatics such as amantadine, memantadine, ribavirin and acyclovir, lipid-lowering agents such as simvastatin or pravastatin, H2 blockers such as ranitidine or famotidine, antibiotics such as macrolides: erythromycin, azithromycin, clarithromycin, roxithromycin, tetracyclines such as doxycycline, minocycline, tetracycline, such as gyrase inhibitors: ciprofloxacin, ofloxacin, β-lactams: such as penicillins, e.g. phenoxyphenylpenicillin, cephalosporins, e.g. cefaclor, cefalexin, cefadroxil, cefixime and inhibitors, e.g. sulbactam, sultamicillin, clavulanic acid, aminoglycosides such as gentamycin, nitroimidazole derivatives such as metamizole ACE inhibitors such as enalapril or amlodipine, immuno-modulators such as azathioprine, methotrexate, cyclo-sporin, tacrolismus, diclizimab and infliximab, calcium channel blockers such as nifedipine, nimodipine and nircardipine, beta blockers such as atenolol, betaxolol, metoprolol, oxprenolol, nebvolol and propranolol, peptides or hormones such as pancreatin, an insulin, a human growth hormone (hGH), corbaplatin, intron A, calcitonin, cromalyn, an interferon, a calcitonin, granulocyte colony stimulating factor (G-CSF), an interleukin, parathyroid hormones, glucagon, pro-somatostatin, a somatostatin, detirelix, cetrorelix, vasopressin, 1-deaminocysteine-8-D-arginine-vasopressin, leuprolide acetate or an antigen which has been isolated from grasses or other plants such as, for example, rye, wheat, barley, oats, bermuda grass, horsetail, sycamore, elm, oak, plane tree, poplar, cedar, horsetail, thistles.

The active substances can if desired also be used in the form of their pharmaceutically acceptable salts or derivatives, and in the case of chiral active substances it is possible to employ both optically active isomers and racemates or mixtures of diastereoisomers. If desired, the compositions of the invention may also comprise two or more pharmaceutical active substances.

Outer Coating c)

The outer coating c) consists substantially of (meth)acrylate copolymers which consist of 40 to 95% by weight free-radical polymerized units of C₁- to C₄-alkyl esters of acrylic or of methacrylic acid and 5 to 60% by weight (meth)acrylate monomers with an anionic group in the alkyl radical. The amounts mentioned normally add up to 100% by weight. However, it is possible in addition, without this leading to an impairment or change in the essential properties, for small amounts in the range from 0 to 10, e.g. 1 to 5, % by weight of further vinylically copolymerizable monomers such as, for example, methyl methacrylate, butyl methacrylate, butyl acrylate or hydroxyethyl methacrylate to be present.

C₁- to C₄-Alkyl esters of acrylic or methacrylic acid are in particular methyl methacrylate, ethyl methacrylate, butyl methacrylate, methyl acrylate, ethyl acrylate and butyl acrylate.

A (meth)acrylate monomer with an anionic group in the alkyl radical may be for example acrylic acid, but preferably methacrylic acid. The carboxyl groups may be partially neutralized to the extent of 30 mol %, preferably to the extent of 5 to 15 mol %.

Suitable anionic (meth)acrylate copolymers are those composed of 40 to 60% by weight methacrylic acid and 60 to 40% by weight methyl methacrylate or 60 to 40% by weight ethyl acrylate (Eudragit® L or Eudragit® L 100-55 types).

Likewise suitable are anionic (meth)acrylate copolymers composed of 20 to 40% by weight methacrylic acid and 80 to 60% by weight methyl methacrylate (Eudragit® S type).

Equally suitable are anionic (meth)acrylate copolymers composed of 20 to 34% by weight methacrylic acid and/or acrylic acid, 20 to 69% by weight methyl acrylate, 0 to 40% by weight ethyl acrylate and, where appropriate, 0 to 10% by weight further vinylically copolymerizable monomers, with the proviso that the glass transition temperature of the copolymer as specified in ISO 11357-2, subsection 3.3.3, does not exceed 60° C. (Eudragit® type with medium content of methacrylic acid).

The copolymer is composed in particular of free-radical polymerized units of

20 to 34, preferably 25 to 33, particularly preferably 28 to 32, % by weight methacrylic acid or acrylic acid, with preference for methacrylic acid,

20 to 69, preferably 35 to 65, particularly preferably to 55, % by weight methyl acrylate and, where appropriate,

0 to 40, preferably 5 to 35, particularly preferably 15 to 35, % by weight ethyl acrylate, with the proviso that the glass transition temperature of the copolymer (without added plasticizer) as specified in ISO 11357-2, subsection 3.3.3, is no higher than 60, preferably 40 to 60, particularly preferably 45 to 55° C.

Also particularly suitable for example are (meth)acrylate copolymers consisting of 10 to 30% by weight methyl methacrylate, 50 to 70% by weight methyl acrylate and 5 to 15% by weight methacrylic acid (Eudragit® FS type).

The dosage form of the invention with said outer coatings, in particular with the type consisting of 10 to 30% by weight methyl methacrylate, 50 to 70% by weight methyl acrylate and 5 to 15% by weight methacrylic acid (Eudragit® FS type) is particularly suitable for dosage forms which release the active substance in the distal ileum or colon.

The dosage form of the invention with said outer coatings, in particular with the type consisting of 10 to 30% by weight methyl methacrylate, 50 to 70% by weight methyl acrylate and 5 to 15% by weight methacrylic acid (Eudragit® FS type) is particularly suitable for dosage forms which comprise the following active substance classes and active substances and can be employed for the therapy of Crohn's disease or of ulcerative colitis. The active substance classes which should be mentioned are those of amino salicylates, of sulfonamides or of glucocorticoids. Particularly preferred active substances are 5-aminosalicylic acid, olsalazine, sulfalazine, prednisone, prednisolone or budesonide.

The dosage form is particularly suitable for immunomodulatory active substances from the classes of protein, peptide, oligonucleotide substances with a presumed site of action on the intestinal mucosa and specifically on the “Payer's patches” in the colonic mucosa.

The dosage form of the invention with said outer coatings, in particular with the type consisting of 10 to 30% by weight methyl methacrylate, 50 to 70% by weight methyl acrylate and 5 to 15% by weight methacrylic acid (Eudragit® FS type) is particularly suitable for dosage forms which comprise the following active substance classes and active substances. The active substance classes which should be mentioned are enzymes, a peptide hormones, immunomodulatory proteins, antigens, antibodies or oligonucleotides.

Particularly preferred active substances are pancreatin, insulin, human growth hormone (hGH), corbaplatin, intron A, calcitonin, cromalyn, interferons, calcitonin, granulocyte colony stimulating factor (G-CSF), interleukin, parathyroid hormones, glucagon, pro-somatostatin, somatostatin, detirelix, cetrorelix, vasopressin, 1-deaminocysteine-8-D-arginine-vasopressin, leuprolide acetate or an antigen which has been isolated from grasses or other plants such as, for example, rye, wheat, barley, oats, bermuda grass, horsetail, sycamore, elm, oak, plane tree, poplar, cedar, horsetail, thistles.

The (meth)acrylate copolymer of the outer coating c) preferably consists substantially to exclusively of the monomers methacrylic acid, acrylic acid, methyl methacrylate, methyl acrylate and/or ethyl acrylate in the proportionate amounts indicated above. The amounts mentioned normally add up to 100% by weight. However, it is possible in addition, without this leading to an impairment or change in the essential properties, for small amounts in the range from 0 to 10, e.g. 1 to 5, % by weight of further vinylically copolymerizable monomers such as, for example, butyl methacrylate, butyl acrylate or hydroxyethyl methacrylate to be present.

Said copolymers can be obtained in a manner known per se by free-radical bulk, solution, bead or emulsion polymerization. Before processing, they must be brought by suitable grinding, drying or spraying processes into the particle size range of the invention.

This can take place by simple crushing of extruded and cooled pellets or hot cut.

The (meth)acrylate copolymer for the outer coating c) may be in the form of and processed as organic solution or as dispersion.

The (meth)acrylate copolymer for the inner coating b) is preferably employed in the form of a dispersion with a solids content of 10-70% by weight.

The (meth)acrylate copolymer c) is preferably in the form of a dispersion, e.g. with a water content of from to 80% by weight. The carboxyl groups may be partially neutralized to the extent of 30 mol %, preferably to the extent of 5 to 15 mol %, by a base, e.g. NaOH.

The inner layer b) is preferably produced by aqueous spraying of an active substance-containing (meth)acrylate copolymer dispersion onto cores, e.g. sucrose pellets, with binding of the active substance after evaporation or sublimation of the water. The product temperature during the spray application can in this connection be for example from 20 to 40, preferably 25 to 35° C. It is usually unnecessary to add a release agent, e.g. talc, and a plasticizer, e.g. triethyl citrate, to the active substance-containing (meth)acrylate copolymer dispersion. Processing of the active substance can preferably take place by stirring into water with initial vigorous mixing, e.g. by mixing for 5 to 15 minutes for example with a high-speed mixer (homogenizer). The suspension obtained in this way can then be added to the (meth)acrylate copolymer dispersion. The mixture should expediently, and preferably also during the spraying process, be agitated continuously.

The layer thickness of the inner coating can preferably be 10-300 μm.

Various Excipients

Release Agents

Release agents have the following properties: they have large specific surface areas, are chemically inert, are free-flowing and comprise fine particles. Because of these properties, they can advantageously be dispersed homogeneously in melts and reduce the tack of polymers which comprise highly polar comonomers as functional groups.

Examples of dryers are:

Alumina, magnesium oxide, kaolin, talc, silica (Aerosils), barium sulfate, carbon black and cellulose.

Further examples of release agents are:

esters of fatty acids or fatty amides, aliphatic, long-chain carboxylic acids, fatty alcohols and esters thereof, montan waxes or paraffin waxes and metal soaps; particular mention should be made of glycerol monostearate (GMS), stearyl alcohol, glycerol behenic acid ester, cetyl alcohol, palmitic acid, canauba wax, beeswax etc.

The inner layer b) usually comprises not more than 1% by weight, preferably no, release agent. If a release agent is used, glycerol monostearate is preferred.

Plasticizers:

Substances suitable as plasticizers ordinarily have a molecular weight between 100 and 20 000 and contain one or more hydrophilic groups in the molecule, e.g. hydroxyl, ester or amino groups. Citrates, phthalates, sebacates, castor oil are suitable. Examples of suitable plasticizers are alkyl citrates, propylene glycol, glycerol esters, alkyl phthalates, alkyl sebacates, sucrose esters, sorbitan esters, diethyl sebacate, dibutyl sebacate and polyethylene glycols 4 000 to 20 000. Preferred plasticizers are tributyl citrate, triethyl citrate (TEC), acetyl triethyl citrate, dibutyl sebacate and diethyl sebacate. The amounts used in the outer layer c) can be between 0 and 35, preferably 2 to 10, % by weight based on the (meth)acrylate copolymer. The inner layer b) usually comprises not more than 20% by weight, preferably not more than 12% by weight and particularly preferably no plasticizer.

Further Excipients Customary in Pharmacy:

Mention should be made here of, for example, stabilizers, colorants, antioxidants, wetting agents, pigments, gloss agents etc. They are used in particular as processing aids and are intended can be to ensure a reliable and reproducible production process and good long-term storage stability. Further excipients customary in pharmacy may be present in amounts of from 0.001% by weight to 30% by weight, preferably 0.1 to 10% by weight, based on the copolymer.

Release Profiles in Hypotonic and Isotonic Medium

The multilayer dosage form of the invention has the property in particular that the values for the percentage release of active substance in a hypotonic and an isotonic release medium based on phosphate buffer pH 6.8 do not differ from one another at any time in the period from 1 to 5 hours by more than 10%, preferably by more than 5%. It is possible to use as hypotonic medium phosphate buffer pH 6.8 with an osmotic concentration of 80 Osmol. The isotonic medium which can be used is phosphate buffer pH 6.8 for which an osmotic concentration of 300 Osmol is adjusted by adding NaCl.

The multilayer dosage form can further be characterized in that in the USP release test two hours at pH 1.2 and a subsequent change in the buffer to pH 7.0 the release of the active substance present is less than 5% in the period up to 2.0 hours after the start of the test and 30 to 100% at the time eight hours after the start of the test.

### EXAMPLES

### Example 1-3: Description of Experiments on Spray Embedding of Budesonide in Eudragit® NE 30 D (Copolymer of 65% by Weight Ethyl Acrylate and 35% by Weight Methyl Methacrylate)

It was investigated whether a delay of release which satisfies therapeutic requirements can be achieved by spray embedding. The formulations were for this purpose varied in the active substance-polymer ratio and the amounts of polymer applied. Specifically, the following polymer to budesonide ratios were produced: 2.5:1 and 1.6:1.

All these formulations were provided with a 3% (m/m) polymer application. In Example 1 (Eudragit® NE 30 D: budesonide 2.5:1) and Example 2 (Eudragit® NE 30 D: budesonide 1.6:1), a sample was in each case taken with a 1% and 2% polymer application. All the batches were mixed with 0.5% Aerosil 200 after production in order to prevent adhesion of the pellets during storage. It is presumed that the active substance budesonide acts as release agent. The release effect of budesonide was examined by completely dispensing with the use of talc as release agent in Example 3 (Eudragit® NE 30 D: budesonide 1.6:1).

Process Conditions/Formulas:

Budesonide fixing by spray embedding with Eudragit® NE 30D

(Investigation of the effects of the polymer: budesonide ratio in the polymer-active substance embedding). Weights in grams.

| | Example | | |
|---|---|---|---|
| | 1 | 2 | 3 |
| Eudragit ® NE 30D | 40 | 40 | 13.3 |
| Budesonide | 4.8 | 7.5 | 2.4 |
| Talc | 6 | 6 | — |
| Water | 166.2 | 159 | 55.4 |
| Total | 217 | 212.5 | 71.1 |
| Polymer: budesonide ratio | 2.5:1 | 1.6:1 | 1.6:1 |
| Sample taken at % polymer application | 1.2 | 1.2 | — |
| Coating dry matter (CDM) [g] | 12 | 12 | 4 |
| Plasticizer based on CDM | — | — | — |
| Release agent based on CDM | 50% | 50% | — |
| Solids content of dispersion (m/m) | 10.5% | 12.0% | 9.0% |
| CDM based on core mass | 3% | 3% | 1% |
| Coating apparatus | Strea 1 | Strea 1 | Strea 1 |
| Nozzle diameter [mm] | 0.8 | 0.8 | 0.8 |
| Spraying pressure [bar] | 0.5 | 0.5 | 0.5 |
| Batch size [g] | 400 | 400 | 400 |
| Amount applied [g] | 217 | 212.5 | 71.1 |
| Preheating time [min] | 5 | 5 | 5 |
| Spraying time [min] | 99 | 122 | 50 |
| Inlet air temperature [° C.] | 27 | 27 | 27 |
| Outlet air temperature [° C.] | 23 | 23 | 23 |
| Spraying rate [g/min] | 2.19 | 1.74 | 1.42 |
| After-drying time [min] | 5 | 5 | 5 |

### Example 4 (Coating for Controlling Release in the Colon)

Process conditions/formulas: Coating experiments with Eudragit® FS 30D (copolymer of 65% by weight methyl acrylate, 25% by weight methyl methacrylate and 10% by weight methacrylic acid) on slow-release budesonide pellets with NE active substance embedding. (Weights in grams).

| | Example |
|---|---|
| | 4 |
| Initial batch | Experiment 1 |
| Eudragit ® FS 30D | 233.3 |
| GMS | 3.5 |
| TEC | 3.5 |
| Tween 80 | 1.4 |
| Water | 191.0 |
| Total | 435.56 |
| Sample at % CDM | 10; 15 |
| Coating dry matter (CDM) [g] | 70 |
| Plasticizer based on CDM [%] | 5.0 |
| Release agent based on CDM | 5.0% |
| Solids content of dispersion (m/m) [%] | 18.0 |
| CDM based on core mass | 20% |
| Coating apparatus | Strea 1 |
| Nozzle diameter [mm] | 0.8 |
| Spraying pressure [bar] | 0.5 |
| Batch size [g] | 350 |
| Amount applied [g] | 435.6 |
| Preheating time [min] | 5 |
| Spraying time [min] | 150 |
| Inlet air temperature [° C.] | 41 |
| Outlet air temperature [° C.] | 30 |
| Spraying rate [g/min] | 3.0 |
| After-drying time [min] | 5 |

### Example 5: (Coating for Controlling Release in the Intestine)

Process Conditions/Formula:

Gastro-resistant coating with Eudragit® L 30 D-55 (copolymer of 50% by weight ethyl acrylate and 50% by weight methacrylic acid) on slow-release budesonide pellets. Weighings took place in grams.

| | Example |
|---|---|
| | 5 |
| Initial batch | Experiment 3 |
| Eudragit ® L 30D-55 | 233 |
| Talc | 35 |
| TEC | 7 |
| Water | 285 |
| Total | 560 |
| Sample at % CDM | 10%, 15% |
| Coating dry matter (CDM) [g] | 70 |
| Plasticizer based on CDM | 10% |
| Release agent based on CDM | 50% |
| Solids content of dispersion (m/m) | 20.0% |
| CDM based on core mass | 20% |
| Coating apparatus | Strea 1 |
| Nozzle diameter [mm] | 0.8 |
| Spraying pressure [bar] | 0.5 |
| Batch size [g] | 350 |
| Amount applied [g] | 560 |
| Preheating time [min] | 5 |
| Spraying time [min] | 248 |
| Inlet air temperature [° C.] | 44 |
| Outlet air temperature [° C.] | 31 |
| Spraying rate [g/min] | 2.56 |
| After-drying time [min] | 5 |

Result of the Investigation

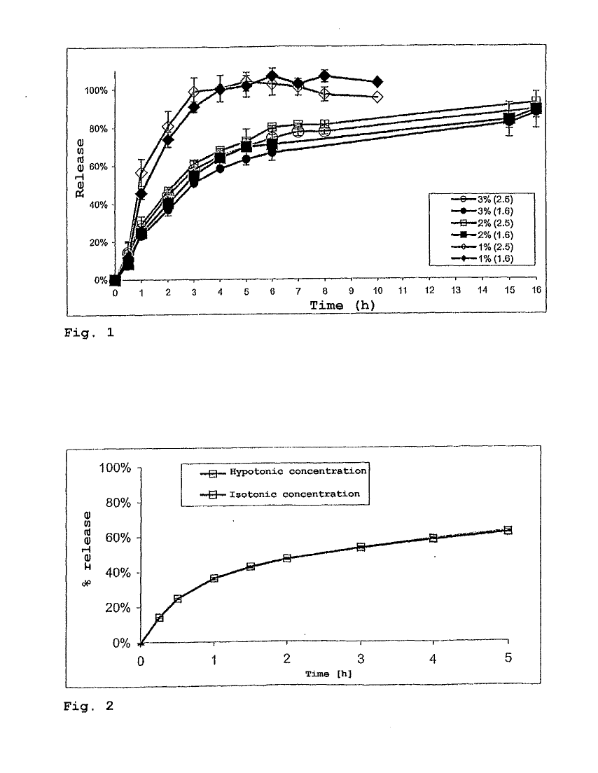

FIG. 1 shows the comparative release profiles of Example 1 (Eudragit® NE 30 D: budesonide 2.5:1) and Example 2 (Eudragit® NE 30 D: budesonide 1.6:1) in phosphate buffer pH 6.8. The release rate decreases with increasing amount of polymer applied, meaning the same as increasing film thickness. The release for 1% polymer applications proceeds quantitatively within 3-4 hours. With a larger CDM application it is possible to observe a reduction in the release rate.

After an accelerated release rate at the start of release, the profiles change into linear release kinetics with a lower rate of release. Examples 1 and 2 with respectively 2% and 3% (m/m) polymer application released between 87.2% and 92.5% of the dose after 16 hours.

With a very small amount applied (1% m/m coating dry matter (CDM)) no homogeneously closed film is to be expected. The active substance is on the contrary present fixed to the polymer, in a very “loose” network on the surface of the nonpareils. The active substance presented on the surface of the matrix is in direct contact with the dissolution medium. Suspended budesonide in the matrix must on the other hand first diffuse after dissolution through the polymer structure in order to reach, following the concentration gradient, the surrounding release medium. Since the ratio of the surface to the polymer matrix is higher with a lower polymer application, it is possible to explain the increased initial release thereby. A larger proportion of the active substance dose is present on the surface of the matrix and is relatively rapidly released. The slowing of release with 3% compared with 2% might be explained by a significant increase in the mass of the polymer matrix, and as a consequence also the thickness of the matrix, when the polymer application is increased, whereas the surface is scarcely affected. The average diffusion pathway becomes longer and the release consequently becomes slower.

FIG. 1 Release profiles of two batches differing in polymer:active substance ratio (2.5:1, Experiment 1) and (1.6:1, Experiment 2) with different amounts of polymer applied in phosphate buffer pH 6.8. The quotient of the respective polymer:active substance ratio is put in round brackets in the key.

Robustness of the Release Behavior

3% polymer coatings of Example 1 (Eudragit NE 30 D:budesonide 2.5:1) were investigated for their robustness in relation to the osmotic concentration of the release medium. Phosphate buffer pH 6.8 with an osmotic concentration of 80 mOsmol and 300 mOsmol was used as dissolution medium. An approximately isotonic concentration of 300 mOsmol was adjusted by adding NaCl to the buffer. This osmolarity range covers the preprandial conditions in the proximal GI tract with and without simultaneous intake of the pellets with up to 250 ml of water. It was observed that the osmolarity had no effect on release from the pellets. Release proceeds in a very robust fashion (FIG. 2).

FIG. 2: Release profiles of Example 1 with 30 (m/m) polymer applied in phosphate buffer and an isotonic and hypotonic osmolarity.

### Example 4: Modification of the Start of Release by a Film Coating with Eudragit® FS 30 D

The coating batch corresponds to Example 1 (spray embedding of budesonide in Eudragit NE 30 D, polymer:active substance ratio 2.5:1, 3% m/m CDM) was coated with Eudragit FS 30 D to modify the start of release. The resulting batch (2-24) was investigated in more detail for its in vitro release behavior. The aim was to slow release of budesonide, the intention being that release starts only in the terminal small intestine.

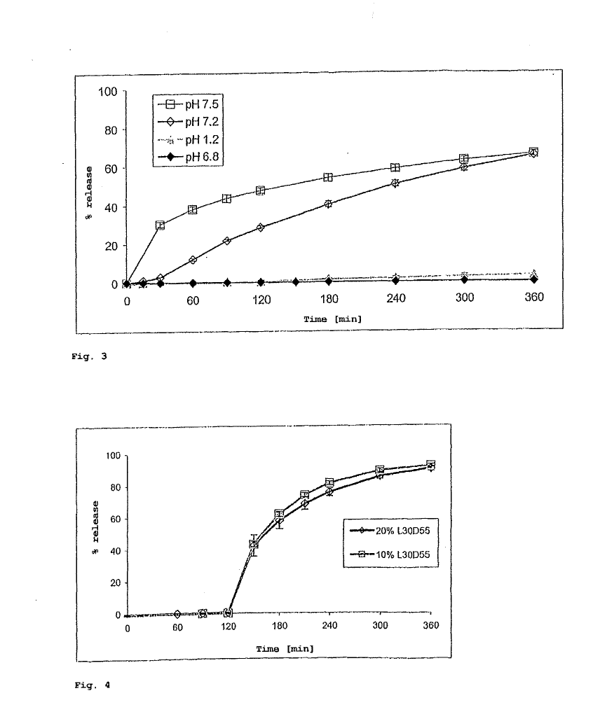

The release investigations carried out in pharmacopeia buffers with pH 1.2, 6.8, 7.2 and 7.5 show a suppression of release at pH 1.2 and 6.8 for Experiment 4 with 20% (m/m) CDM of Eudragit® FS 30 D. That is to say at pH values intended to simulate the stomach and the proximal small intestine. Release starts, with a short t_{lag} phase of between 15 to 30 minutes, in buffer of pH 7.2. Release then follows a slow, almost linear course. The outer polymer does not yet dissolve in at this pH, but the swelling is very pronounced. Release is in this case controlled by diffusion through the swollen polymer. At pH 7.5, release starts immediately without a lag time being observable. The outer polymer Eudragit® FS 30 D dissolves rapidly, and release is controlled solely by the embedding of active substance in Eudragit® NE 30 D (FIG. 3).

FIG. 3. Release profiles of Experiment 4 (20% (m/m) Eudragit® FS 30 D coating on budesonide spray embedding in Eudragit® NE 30 D (polymer:active substance ratio 2.5:1)) in pharmacopoeia buffers with different pH values.

### Example 5: Gastro-Resistant Coating with Eudragit® L 30 D-55

Experiment 5 was selected as prototype for the therapy of Crohn's disease and characterized in detail by in vitro release investigations. The batch is composed of a spray embedding of budesonide in Eudragit® NE 30 D with 1% (m/m) CDM applied and a gastro-resistant coating polymer, namely Eudragit® L 30 D-55 with 10% or 20% CDM applied. Since the gastro-resistant polymer coating is in direct contact with the embedding matrix, it was of interest to examine a possible influence of the coating on the release from the embedding. The test for resistance to gastric juice was carried out according to the USP 24 monograph “Delayed-release (Enteric-coated) Articles—General Drug Release Standard”, Method A. There was no measurable release in simulated gastric fluid over 2 hours either with 10% or 20% (m/m) polymer applied. After buffering to pH 6.8, release started without delay. It was observed that the release profile is scarcely influenced by the higher, 20% polymer application compared with the 10% CDM application.

FIG. 4. Release profile of Example 5 with 10% and 20% (m/m) Eudragit® L 30 D-55 polymer applied. Release for 2 hours in 0.1 N HCl and then change of buffer to pH 6.8.

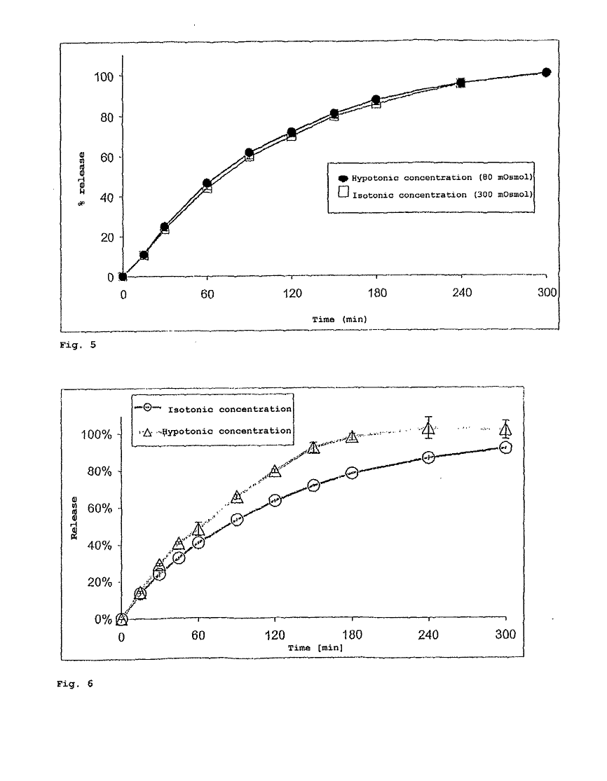

FIG. 5: Release profiles of Experiment 5 in dissolution medium (phosphate buffer, Ph 6.8) differing in osmolarity. Embedding in Eudragit® NE 30 D (Experiment 3) and coating with Eudragit® L 30 D (copolymer of 50% by weight methyl methacrylate and 50% by weight methacrylic acid).

The release investigation, the results of which are depicted graphically in FIG. 5, shows that the release from the pellet behaves very robustly in respect of changes in the release medium. An osmotic effect on the release profile in the range from 80 to 300 mOsmol is practically undetectable in the experiments.

Example 6 (not According to the Invention, Comparison with Example 1)

Formulation comparison with Example 1.) with Eudragit RL 30D as release-slowing coating and Eudragit® L 30 D-55 as gastro-resistant film coating (GR). Weights in grams.

Example 6

| | Slow | GR |
|---|---|---|
| Eudragit RL 30D | 200 | — |
| Eudragit L 30D-55 | — | 167 |
| Talc | 30 | 25 |
| TEC | 12 | 5 |
| Water | 268 | 203 |
| Total | 510 | 400 |
| Coating dry matter (CDM) [g] | 60 | 50 |
| Plasticizer based on CDM | 20% | 10% |
| Release agent based on CDM | 50% | 50% |
| Solids content of dispersion (m/m) | 20.4% | 20% |
| CDM based on core mass | 12% | 20% |
| Coating apparatus | Strea 1 | Strea 1 |
| Nozzle diameter [mm] | 0.8 | 0.8 |
| Spraying pressure [bar] | 0.5 | 0.5 |
| Batch size [g] | 500 | 250 |
| Amount applied [g] | 510 | 400 |
| Preheating time [min] | 5 | 5 |
| Spraying time [min] | 213 | 167 |
| Inlet air temperature [° C.] | 34 | 40 |
| Outlet air temperature [° C.] | 25 | 30 |
| Spraying rate [g/min] | 2.4 | 2.4 |
| After-drying time [min] | 5 | 10 |

FIG. 6: Release profiles of Example 6 aminosalicylic acid pellets with a release-slowing coating of Eudragit RL 30 D and a gastro-resistant coating of Eudragit L 30 D-55 in phosphate buffer differing in osmolarity.

All release tests were carried out by USP method 2 (Paddle) with a speed of 100 rpm.

1-12. (canceled)

13. A multilayer dosage form, comprising
a) a neutral core;
b) at least one inner layer comprising at least a portion of a pharmaceutically active substance and a methacrylate copolymer, wherein said methacrylate copolymer comprises at least 90% by weight of (meth)acrylate monomers having neutral radicals and has a minimum film-forming temperature as specified in DIN 53 787 not exceeding 30° C., and wherein the pharmaceutically active substance is in bound form; and
c) an outer layer comprising Eudragit® L 30D;
wherein values for the percentage release of the pharmaceutically active substance from the multilayer dosage form in a hypotonic and an isotonic release medium based on phosphate buffer pH 6.8 do not differ from one another at any time in the period from 1 to 5 hours by more than 10%.

14. The multilayer dosage form of claim 13, wherein the values for the percentage release of the pharmaceutically active substance from the multilayer dosage form in the hypotonic and the isotonic release medium do not differ from one another at any time in the period from 1 to 5 hours by more than 5%.

15. The multilayer dosage form of claim 13, wherein the hypotonic release medium based on phosphate buffer pH 6.8 has an osmotic concentration of about 80 Osmol/L.

16. The multilayer dosage form of claim 13, wherein the isotonic release medium based on phosphate buffer pH 6.8 has an osmotic concentration of about 300 Osmol/L.

17. The multilayer dosage form of claim 13, wherein the at least one inner layer has a thickness of 10-300 μm.

18. The multilayer dosage form of claim 13, wherein the pharmaceutically active substance is methylphenidate or a pharmaceutically acceptable salt thereof.
